(19) United States
(12) Plant Patent Application Publication
(10) Pub. No.: US 2017/0374775 P1
(43) Pub. Date: Dec. 28, 2017

(54) Apple Tree Named 'GALA BIGBUCKS'

(50) ;

(71) Applicant: Pink Vein (Pty) Ltd., Elgin (ZA)

(72) Inventors: Derek Alexander Corder, Elgin (ZA); Peter John Nel, Somerset West (ZA); Anthony George Rawbone-Viljoen, Elgin (ZA)

(73) Assignee: Pink Vein (Pty) Ltd., Elgin (ZA)

(21) Appl. No.: 14/999,781

(22) Filed: Jun. 27, 2016

(51) Int. Cl. A01H 5/08

(52) U.S. Cl. CPC A01H 5/0875
USPC PLT/162

(57) ABSTRACT

A new and distinct Malus domestica Borkh. apple tree named ‘GALA BIGBUCKS’, particularly characterized by its dark-red overcolor on 90-100% of fruit skin surface; foliage with distinct red midrib at harvest time; red/purple fruit stem; and intense red flower filament base.

Latin name of the genus and species of the plant claimed: Malus domestica Borkh.

Variety denomination: GALA BIGBUCKS.

### BACKGROUND OF THE INVENTION

The present invention relates to a new and distinct variety of Apple tree, botanically known as Malus domestica Borkh. of the Rosaceae family, and hereinafter referred to by the variety denomination ‘GALA BIGBUCKS’.

The new Malus variety was discovered and selected by the inventors as a naturally-occurring mutation growing in a cultivated orchard in 2011 in Elgin, South Africa. The new Malus variety was selected by the inventors based on its superior dark wine-red blush overcolor. Asexual reproduction of the new Malus variety by budding was first performed in February 2012 in Riviersonderend, South Africa, and has demonstrated that the combination of characteristics as herein disclosed for the new variety are firmly fixed and retained through successive generations of asexual reproduction. The new variety reproduces true to type.

### BRIEF DESCRIPTION OF THE INVENTION

The following traits have been repeatedly observed and are determined to be unique characteristics of ‘GALA BIGBUCKS’ which in combination distinguish this apple tree as a new and distinct variety:
1. Dark red overcolor on 90-100% of fruit skin surface;
2. Foliage with distinct red/purple midrib at harvest time;
3. Red/purple fruit stem; and
4. Intense red flower filament base.

Of the many commercial varieties known to the present inventor(s), the most similar in comparison to the new Malus variety ‘GALA BIGBUCKS’ is the Malus variety ‘ROYAL BEAUT GALA’ (unpatented), in the following characteristics described in Table 1:

TABLE 1

| | | Comparison Variety ‘ROYAL |
|---|---|---|
| | New Variety ‘GALA | BEAUT GALA’ |
| Characteristic | BIGBUCKS’ | (unpatented) |
| Overcolor | Red flush | Striated Red/Pink |
| Color of Leaf | Red/purple | Green |
| Midrib | | |
| Color of Fruit | Red/purple | Green |
| Stem | | |
| Color of Base of | Red | Pale Green |
| Flower Filament | | |

### BRIEF DESCRIPTION OF THE PHOTOGRAPHS

The accompanying photographs, in combination with the brief description given, illustrate the overall appearance of the new Malus variety ‘GALA BIGBUCKS’ showing the colors as true as is reasonably possible with colored reproductions of this type.

Colors in the photographs may differ slightly from the color values cited in the detailed botanical description, which accurately describe the color of ‘GALA BIGBUCKS’.

FIG. 1 shows a typical fruit of ‘GALA BIGBUCKS’ at harvest time.

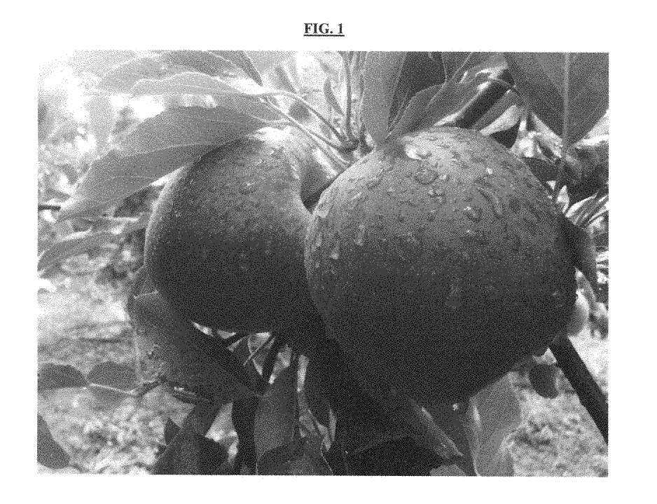

FIG. 2 shows a typical fruit of ‘GALA BIGBUCKS’ 14 days before harvest time (left), compared with a typical fruit of ‘ROYAL BEAUT GALA’ 14 days before harvest time (right).

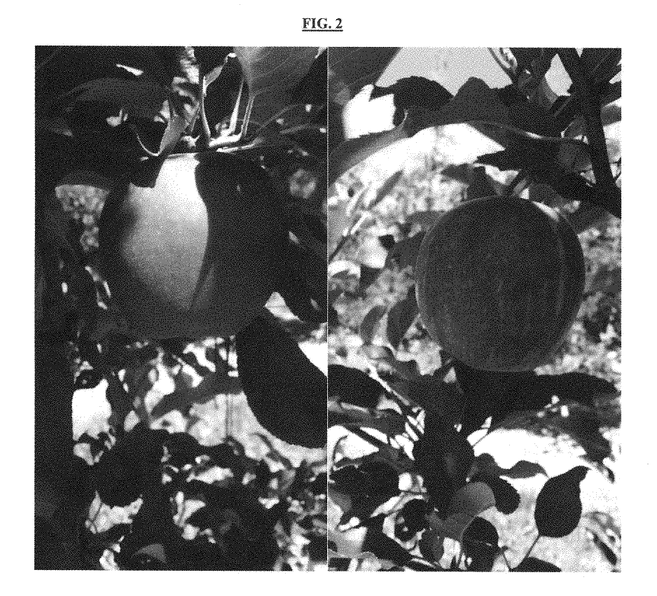

FIG. 3 shows the bottom part of the filament of the flower of ‘GALA BIGBUCKS’ (left), compared with the bottom part of the filament of the flower of ‘ROYAL BEAUT GALA’ (right).

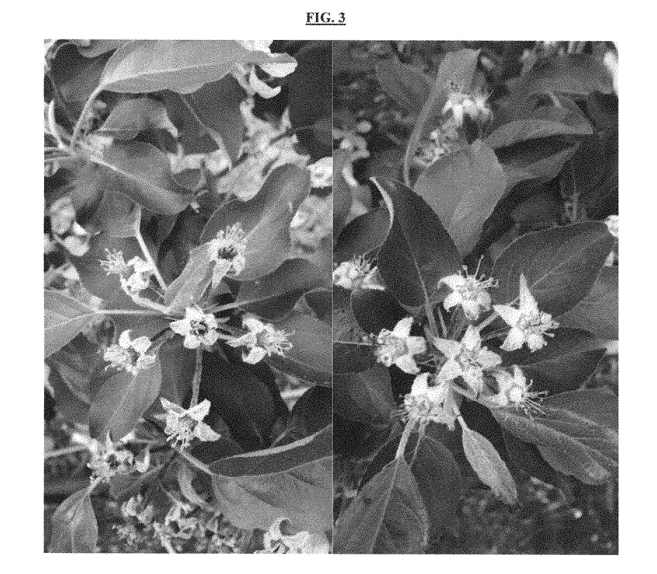

FIG. 4 shows a typical whole fruit 30 days before harvest of ‘GALA BIGBUCKS’ (right), showing a red/purple stem, compared with a typical whole fruit of ‘ROYAL BEAUT GALA’ (left), showing a green stem.

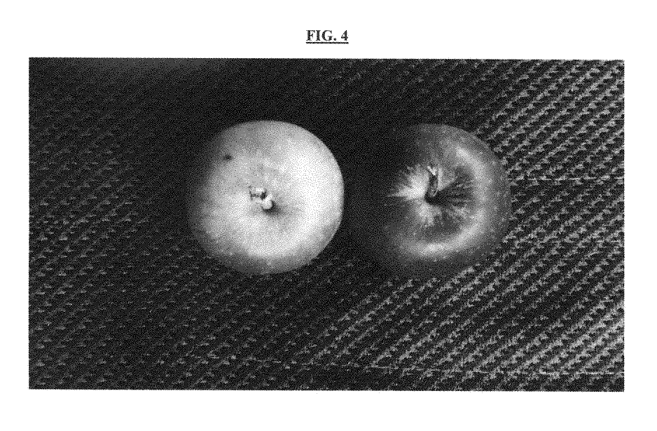

FIG. 5 shows the difference in color of the fruitlet (7 mm) of ‘ROYAL BEAUT GALA’ (top), and ‘GALA BIGBUCKS’ (bottom).

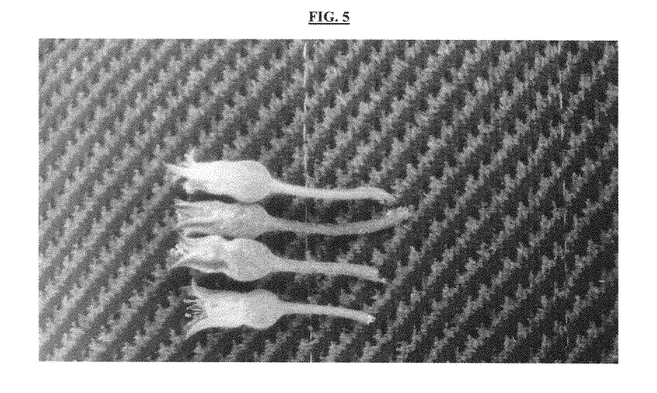

FIG. 6 shows the difference in color of the midrib on the under surface of a typical leaf of ‘GALA BIGBUCKS’ (left), and ‘ROYAL BEAUT GALA’ (right).

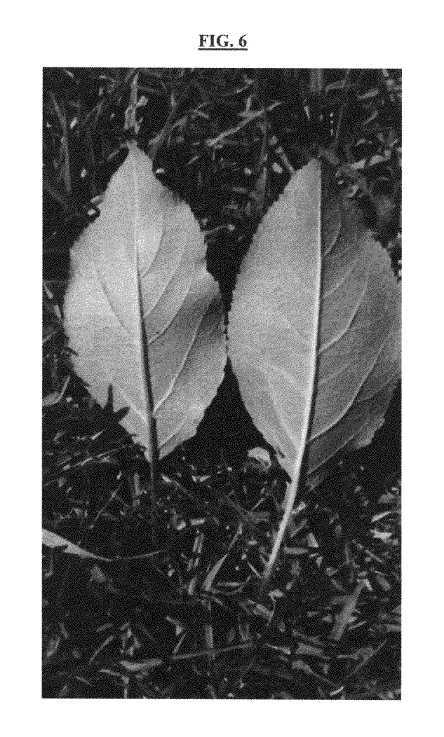

### DETAILED BOTANICAL DESCRIPTION

The new Malus variety ‘GALA BIGBUCKS’ has not been observed under all possible environmental conditions. The phenotype of the new variety may vary with variations in environment such as temperature, light intensity, and day length without any change in the genotype of the Apple tree.

The aforementioned photographs, together with the following observations, measurements and values describe trees of ‘GALA BIGBUCKS’ as grown in the Apple farm in Elgin, South Africa and Die Eike, Ceres, South Africa, under conditions which closely approximate those generally used in commercial practice; loamy soil with 100 cold units in Elgin, South Africa and loamy soil with 1000 cold units in Ceres, South Africa.

Unless otherwise stated, the detailed botanical description includes observations, measurements and values based on 4 year old ‘GALA BIGBUCKS’ trees grown in the Apple farm in Elgin, South Africa and Die Eike, Ceres, South Africa from 2013 to 2016.

Quantified measurements are expressed as an average of measurements taken from a number of trees of ‘GALA BIGBUCKS’. The measurements of any individual tree, or any group of trees, of the new variety may vary from the stated average.

Color references are made to The Royal Horticultural Society Colour Chart (R.H.S.), (Fifth Edition), except where general colors of ordinary significance are used. Color values were taken under daylight conditions in the morning in Elgin and Ceres, South Africa.

All of the trees of ‘GALA BIGBUCKS’, insofar as they have been observed, have been identical in all the characteristics described below.
Classification:
   Botanical.—Malus domestica Borkh.
Parentage: Naturally-occurring mutation discovered in a cultivated area (orchard).
Propagation: budding.
Growing conditions:
   Light intensities.—Full sunlight or slight shade.
   Temperature.—During day, grown in range of 5° C. to 33° C. Fertilization: Standard for Royal Gala.
   Growth regulators.—none used.
   Pruning or trimming requirements.—Standard for 4 year old Royal Gala.
Tree:
   Age.—Observed trees were 4 years old bearing trees.
   Vigor.—Strong vigor; 2^{nd} leaf beginning production; average productivity and good flowering; good bearing.
   Form.—Ramified and conical with narrow branch angles.
   Habit.—A semi-upright tree with one trunk and 1 leader; main branches spreading; crown symmetrical.
   Branching habit.—Main branches angle is 40° to 90° with respect to trunk if allowed to grow naturally.
   Cropping behavior.—First crop in 2^{nd} leaf; medium production. Abundant flowering and no biennial bearing.
   Type of bearing.—(Spurs and short shoots) Production (2 years): About 5000 Kg per hectare.
   Size at maturity.—Height: About 3 m Spread: About 1.5 m.
   Trunk.—Height (up to leaders): About 900 mm Diameter: About 95 mm.
   Texture.—At first, smooth with numerous lenticels, then shallow furrows and scaly ridges.
   Bark color.—Greyish Brown.
   Branches.—Number per tree: About 12 Color: Mature after about 4 months New Growth: Green Internode length: About 26 mm Internode diameter: About 5 mm Branch lenticels: Length: About 0.9 mm Width: About 0.4 mm Color: Whitish Density: Medium Spur Present: Yes Distance between each spur: About 30 mm Diameter of each spur: About 5 mm Number of fruit per spur: About 2 to 4.
Foliage:
   Arrangement.—Alternate, simple, petiolated Lamina.
   Size.—Length: Medium (90 mm) Width: Very narrow (47 mm) Length/width ratio: Very large (1.91) Overall Shape: Narrow elliptic Base shape: Truncate Apex shape: Right Angled Margin: Serrate type 2 Texture: Upper surface: Smooth Under surface: Pubescence Weak Attitude in relation to shoot: Upwards Color (mature leaves): Upper surface: Green, Group N137C Medium Under surface: Yellow/green 148B; Midrib Red/Purple Group 59A Color (immature leaves).
   Venation.—Color: Red/Purple Group 59A.
   Petiole.—.
   Length.—About 31 mm Diameter: About 1.9 mm.
   Texture.—Smooth.
   Color.—Purple/Red N77A Stipule.
   Arrangement.—At base of petiole Length: 6 mm.
   Width.—About 1 mm.
Inflorescence:
   Blooming time.—Full bloom on about 15 October in Southern Hemisphere.
   Blooming period.—About 10 October to 29 October in Southern Hemisphere.
   Fragrance.—Weak.
   Number of flowers per inflorescence.—About 6.
   Inflorescence size.—Diameter: About 44 mm.
   Petals.—Arrangement: Free Number per flower: Five Overall shape: Narrow elliptic Apex shape: Moderately obtuse Base shape: Acute Texture (upper and lower surface): smooth Margin: undefined Color (upper surface): Red Group 51C.
   Sepals.—Number per flower: Five.
   Pedicel.—Texture: Smooth.
Fruit:
   Keeping quality.—The fruit keeps very well on the tree. It can be stored in cold temperature conditions for up to 6 months without losing firmness and juiciness. It has a shelf life up to 2 weeks without losing firmness and juiciness.
   Maturity when described.—Ripe for eating first week of February in Southern Hemisphere.
   Maturity period after full bloom.—About ±100 days after full bloom; about February 7 in Southern Hemisphere.
   Date of first and last picking (harvest).—About February 1 to February 15 in Elgin, South Africa (34.15432° S, 19.047473° E).
   Type.—Pome.
   General shape.—Cylindrical-globuse.
   Average weight.—About 145 g.
   Fruit size.—Average height: Long (65.2 mm) Average diameter (at widest point): About 71 mm Position of maximum diameter: Middle (½ of height). Height/thickness ratio: Medium (0.91).
   Stem.—Length: About 28 mm Diameter: About 2 mm Color: Red/Purple 59B.
   Stalk cavity.—Depth: About 15 mm Width: About 32 mm.
   Eye basin.—Depth: About 7.6 mm Width: Medium 32.3 mm Crowning at calyx end: Moderate Position of sepals: Partly Open Calyx tube: Funnel form.
   Skin.—Thickness: 0.1 mm Bloom: Weak Greasiness: absent Firmness (at picking time): 7 to 9 kg/cm² Overcolor Color: Red Group 53A Percentage of skin surface with overcolor color: About 90 to 100% Pattern of overcolor: Solid Flush Intensity of overcolor: Dark Ground color: Not visible.
   Flesh.—Color: Cream yellow Group 12C Texture: Crisp and juicy Sugar content (at picking time): 13° Brix Acidity/Starch (at picking time) Starch conversion 30% \.
   Core.—Symmetry of core: Symmetrical Distinctness of core lines: Medium.
   Locules.—Number (per fruit): 5 Length: About 23 mm Width: About 22 mm Form: Moderately open.
Seeds:
   Number per fruit.—About 8.
   Number per locule.—About 1-2.
   Shape.—Ovate.
   Length.—About 7 mm.
   Width.—About 4.5 mm.
   Texture.—Smooth.
   Color.—Greyed-Orange 177A.
Reproductive organs:
   Androecium.—Filaments: Color: Base is intense pink/red Pollen: Amount: Abundant.
Use: Fresh market.
Disease/pest resistance: No resistance to Apple Scab (Venturia inaequalis) observed as for Royal Gala.
Disease/pest susceptibility: No abnormal susceptibility observed (As for ‘Royal Gala.’).
Winter hardiness: No information available in Southern Hemisphere.
Drought/heat tolerance: Tolerant to temperatures up to 40° C., growth is limited by drought periods without irrigation.
Shipping/storage characteristics: not susceptible to bruising (As for ‘Royal Gala’).

I claim:

1. A new and distinct variety of Malus domestica Borkh. apple tree named ‘GALA BIGBUCKS’, as illustrated and described herein.
